(19) United States
(12) Patent Application Publication
(10) Pub. No.: US 2020/0276419 A1
(43) Pub. Date: Sep. 3, 2020

(54) Vascular Access Device and Related Method

(71) Applicant: Prytime Medical Devices, Inc., Boerne, TX (US)

(72) Inventors: Curtis J. FRANKLIN, Lakewood, CO (US); Todd J. KRUMMENACHER, Lakewood, CO (US); Jeremy REYNOLDS, Lakewood, CO (US)

(21) Appl. No.: 16/288,624

(22) Filed: Feb. 28, 2019

(51) Int. Cl. A61M 25/09; A61M 25/06

(52) U.S. Cl. CPC A61M 25/09041; A61M 25/0693; A61M 25/065

(57) ABSTRACT

An access device includes a body, a needle and a guidewire. The body has a guidewire channel extending from a distal end of the body toward the proximal end. The guidewire channel opens into a funnel-like port having a proximal port diameter greater than a distal port diameter. The distal port diameter is substantially the same as a guidewire channel diameter. The needle has a tip, a proximal end and a needle lumen. The proximal end of the needle is removably secured to a distal end of the body in an assembled configuration. A guidewire has a front end portion positioned in the guidewire channel in an initial configuration and a guidewire diameter. The guidewire channel diameter is greater than the guidewire diameter and the guidewire channel facilitates blood flow between the guidewire and an internal surface of the guidewire channel when the guidewire is positioned in the guidewire channel.

### BACKGROUND OF THE INVENTION

Known arterial and vascular access procedures are conducted using a cannula needle and guidewire as distinct and separate components. Vascular access procedures may also utilize a separate syringe that is attached to a connecting portion to draw a vacuum on the needle and withdraw blood from the vein when the needle tip is positioned within the vein. The tip of the needle is inserted through the patient's skin and into the blood vessel (vein or artery), which may be difficult to locate and pierce due to differing patient anatomy. In addition, the proper placement of the needle in the blood vessel may be difficult to confirm due to limited pressure in the blood vessels (particularly in veins), positioning of different blood vessels near each other, patient anatomy and other factors. Appropriately positioning the needle tip in the vein may be facilitated by pulling a vacuum in the needle using a separately attached syringe to draw blood from the vessel, typically a vein, once the tip is positioned in the vessel. This vacuum is typically not required when accessing an artery, as arterial pressure typically causes blood to flow into and out of a proximal end of the needle when the tip is in the artery. When the tip is in the vessel, blood flow visually confirms the appropriate positioning of the needle. Proper positioning may, however, be lost while the medical professional reaches for and attempts to insert a guidewire into the needle or access device. The needle or access device may move such that the tip moves out of the targeted vessel because the patient or needle moves, the medical professional conducts a follow-up step related to the procedure, the medical professional moves the needle or access device while arranging the guidewire or for various other reasons.

Once the tip of the needle is positioned in the blood vessel, the physician or medical technician typically inserts a separate guidewire through the needle and into the blood vessel. The guidewire is typically retrieved from a nearby surgical table or other proximate area. The physician's or medical technician's movement to retrieve the guidewire can result in movement of the tip of the needle, potentially resulting in the tip moving out of the blood vessel. This movement in reaching or otherwise retrieving the guidewire also slows the procedure, which can be important in trauma situations and for patient comfort. Feeding the guidewire into the proximal end of the needle or connector following retrieval can also be difficult, resulting in additional potential for movement of the tip out of the blood vessel and delay in the procedure. For example, the physician may need to re-start the procedure of positioning the tip of the needle in the vessel if the tip moves out of the vessel or may need to re-arrange the tip back into the appropriate vessel, thereby delaying the procedure and often increasing patient discomfort. Feeding the guidewire into the proximal end of the needle is particularly difficult when the needle has a small diameter at its proximal end, the distal end of the guidewire has a J-shape that is preferably straightened before insertion into the proximal end of the needle and/or the medical professional is rushing the procedure, particularly in trauma situations.

Traditional vascular access procedures may be performed in the field using a cannula needle and guidewire as individual components. For some access procedures, typically venous access, a needle is used with a syringe attached to the needle via a needle hub or Luer connection. The needle is inserted into the vessel while drawing back on the syringe plunger to create vacuum so that blood is drawn into the syringe upon access to the vein or as an indication of when the tip of the needle is positioned within the vein (presence of blood indicates the needle is in the vessel). Next, the syringe is removed from the needle, a guidewire is aligned with and inserted into a proximal end of the needle and the guidewire is advanced through the needle into the vessel. The guidewire is urged into the needle such that the guidewire is positioned within the vessel, preferably the vein, as a guide for subsequent instruments. The needle is then removed proximally over the guidewire with a front portion of the guidewire remaining in the vessel and a rear portion of the guidewire extending out of the patient's skin. The guidewire provides a path for insertion of other devices into the vessel and the other devices are typically used for treatment that is associated with the vessel. This multiple step process and exchange of components, including the needle, syringe, other instrument and guidewire may cause pain for the user and provides multiple opportunities for the needle or guidewire to move or become misaligned, potentially requiring the medical professional to re-start the procedure or realign the components, particularly the tip of the needle before the guidewire is inserted into the vessel.

Traditional access procedures using a guidewire also often utilize a cheater, guidewire insertion tool or guidewire straightener into which the distal portion of the guidewire is inserted to straighten the distal portion of the guidewire and to facilitate alignment of the guidewire with the needle and port. The cheater is typically, relatively short, having a length of approximately one inch (1″), and does not include a distal Luer connector that permits connection of the cheater with the proximal end of the needle, which typically has a female Luer connection. The cheater must be held onto the proximal end of the needle by the technician while the guidewire is fed into the needle and the patient. In addition, the relatively short length of the cheater results in a relatively flexible distal portion of the guidewire extending out of the proximal end of the cheater when loaded, which can cause the guidewire to slide or fall out of the cheater unless the technician is holding the guidewire into the cheater. Further, the relatively flexible distal portion of the guidewire may buckle between the proximal end of the cheater and the stiffer portion of the guidewire when the technician initially attempts to urge the guidewire into the needle and patient because the relatively flexible portion of the distal tip of the guidewire is not supported by the cheater. It would be desirable to design, develop and deploy an access device that overcomes these shortcomings of the guidewire cheater and guidewire insertion tool.

Access devices have also been developed that include an integrated hypodermic or vascular access needle, and potentially other components, for use specifically in the access procedure. These access devices are typically limited for use with predetermined guidewire sizes that correlate with the size of the integrated hypodermic or vascular access needle. These devices with integrated hypodermic needles also include internal flow channels that permit flow of flash blood and indicate flash from a side surface or side flashport at the body of the device. These internal channels are difficult to clean and/or sterilize after blood or other fluids flow through the internal channels and are not reusable in most situations following an initial flash or blood flow.

During the standard Seldinger technique, the needle is inserted through the skin and into the blood vessel. Blood flashes from the needle Luer fitting. At this point, the user typically turns away from the needle momentarily to grab the guidewire and straighten the J-tip at the distal end of the guidewire. More often than not, especially for less experienced users, the needle tip has moved during this step and is no longer in the blood vessel and may have punctured the blood vessel at a second location in addition to the original entry puncture. When the user tries to advance the guidewire, it jams because the needle tip is no longer in the blood vessel. The needle must then be removed from the skin, flushed and another attempt made. Cleaning and sterilization are difficult because of the small diameter of the needle lumen.

The preferred access device with a guidewire also addresses the above-described shortcomings of prior art blood vessel access devices and methods. For example, it would be desirable to design, develop and deploy an access device that eliminates the requirement to straighten and insert a very soft guidewire tip into the needle after the tip of the needle is inserted into the patient's vessel to expedite the vessel access procedure. It would also be desirable to design, develop and deploy an access device that is quickly able to assess whether the tip of the access device is positioned in the patient's vessel without significant flash blood flow out of the access device that provides an indication of whether the tip is in an artery or a vein. In addition, it would be desirable to design, develop and deploy an access device that has a built-in holder to secure the proximal end of the guidewire to limit the relatively flexible guidewire from touching non-sterile surfaces in the area around the procedure prior to entering the patient. It would further be desirable to design, develop and deploy an access device that may be flushed free of blood after an access attempt and that is able to adapt to various sized hypodermic or vascular access needles that are readily available to the medical provider.

### BRIEF SUMMARY OF THE INVENTION

Briefly stated, the preferred invention is directed to an access device for accessing a lumen of a blood vessel including a body having a distal end, a proximal end, a longitudinal axis, a guidewire channel, a guidewire holder and a flash channel. The guidewire channel extends generally parallel to the longitudinal axis. The guidewire channel is defined at the distal end of the body at an external surface of the body. A needle has a tip, a proximal end and a needle lumen extending to the tip. The proximal end of the needle has a funnel-like port with an internal surface. The proximal end is removably secured to a distal end of the body in an assembled configuration. The internal surface of the proximal end of the needle and the flash channel define a flash lumen. A guidewire has a front end portion that is positioned in the guidewire channel in an initial configuration.

In another aspect, the preferred invention is directed to an access device for accessing a lumen of a blood vessel. The access device includes a body, a needle and a guidewire. The body has a distal end, a proximal end, a longitudinal axis and a guidewire channel. The guidewire channel extends generally parallel to the longitudinal axis. The guidewire channel is defined at the distal end of the body and extends toward the proximal end of the body. The guidewire channel opens into a funnel-like port positioned at the proximal end. The funnel-like port has a proximal port diameter that is greater than a distal port diameter. The funnel-like port may be comprised of a Luer fitting, potentially a female slip Luer that is able to connect to a male Luer fitting. The distal port diameter is substantially the same as a guidewire channel diameter. The needle has a tip, a proximal end and a needle lumen extending to the tip. The proximal end of the needle has a funnel-like port with an internal surface. The proximal end is removably secured to the distal end of the body in an assembled configuration. The guidewire has a front end portion. The front end portion is positioned in the guidewire channel in an initial configuration. The guidewire has a guidewire diameter. The guidewire channel diameter is greater than the guidewire diameter. The guidewire channel is configured to facilitate blood flow between the guidewire and an internal surface of the guidewire channel when the guidewire is positioned in the guidewire channel.

### BRIEF DESCRIPTION OF THE SEVERAL VIEWS OF THE DRAWINGS

The foregoing summary, as well as the following detailed description of preferred embodiments of the access device and method of the present application, will be better understood when read in conjunction with the appended drawings. For the purposes of illustrating the access device, there are shown in the drawings preferred embodiments. It should be understood, however, that the application is not limited to the precise arrangements and instrumentalities shown. In the drawings:

FIG. 1 is a side perspective view of an access device in accordance with a first preferred embodiment of the present invention;

FIG. 2 is a cross-sectional view of the access device of FIG. 1, taken along line 2-2 of FIG. 1;

FIG. 3 is an alternative side perspective view of the access device of FIG. 1 with a guidewire mounted to the access device;

FIG. 4 is a side perspective, cross-sectional view of the access device of FIG. 1, taken along line 4-4 of FIG. 3;

FIG. 5 is cross-sectional view of the access device of FIG. 1, taken along line 4-4 of FIG. 3;

FIG. 6 is a side perspective view of the access device of FIG. 1 with a hypodermic or vascular access needle mounted to a distal end of the access device;

FIG. 7 is a cross-sectional view of the access device of FIG. 1, taken along line 7-7 of FIG. 6;

FIG. 8 is a magnified, top perspective view of the access device of FIG. 1, taken from within shape 8 of FIG. 6;

FIG. 9 is a cross-sectional view of a portion of the access device of FIG. 1, taken along line 9-9 of FIG. 8;

FIG. 10 is a side perspective view of an access device in accordance with a second preferred embodiment of the present invention;

FIG. 11 is a cross-sectional view of the access device of FIG. 10, taken along line 11-11 of FIG. 10;

FIG. 12 is a side perspective view of an access device in accordance with a third preferred embodiment of the present invention;

FIG. 12A is a side perspective view of the access device of FIG. 12, with an alternative guidewire holder and a guidewire positioned in the body in a loaded configuration;

FIG. 12B is a magnified front perspective view of a distal end of the access device of FIG. 12A;

FIG. 12C is a magnified side elevational view of a body of the access device of FIG. 12A;

FIG. 13 is a cross-sectional view of the access device of FIG. 12, taken along line 13-13 of FIG. 12;

FIG. 14 is a side perspective view of an access device in accordance with a fourth preferred embodiment of the present invention;

FIG. 15 is a cross-sectional view of the access device of FIG. 14, taken along line 15-15 of FIG. 14;

FIG. 16 is a magnified, cross-sectional view of the access device of FIG. 14, taken from within shape 16 of FIG. 15;

FIG. 17 is a side perspective view of an access device in accordance with a fifth preferred embodiment of the present invention;

FIG. 18 is a cross-sectional view of the access device of FIG. 17, taken along line 18-18 of FIG. 17;

FIG. 19 is a magnified, side perspective view of the access device of FIG. 17, taken from within shape 19 of FIG. 17;

FIG. 20 is a magnified, side perspective view of a portion of the access device of FIG. 17, with a proximal portion of a hypodermic or vascular access needle mounted to a distal end of the access device;

FIG. 21 is a side perspective view of an access device in accordance with a sixth preferred embodiment of the present invention;

FIG. 22 is a cross-sectional view of the access device of FIG. 21, taken along line 22-22 of FIG. 21;

FIG. 23 is a side perspective view of an access device in accordance with a seventh preferred embodiment of the present invention;

FIG. 24 is a cross-sectional view of the access device of FIG. 23, taken along line 24-24 of FIG. 23;

FIG. 25 is a side perspective view of an access device in accordance with an eighth preferred embodiment of the present invention; and

FIG. 26 is a cross-sectional view of the access device of FIG. 25, taken along line 26-26 of FIG. 25.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one”. The words “right”, “left”, “lower” and “upper” designate directions in the drawings to which reference is made. The words “inwardly” or “distally” and “outwardly” or “proximally” refer to directions toward and away from, respectively, the patient's body, or the geometric center of the preferred access device and related parts thereof. The words, “anterior”, “posterior”, “superior,” “inferior”, “lateral” and related words and/or phrases designate preferred positions, directions and/or orientations in the human body to which reference is made and are not meant to be limiting. The terminology includes the above-listed words, derivatives thereof and words of similar import.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

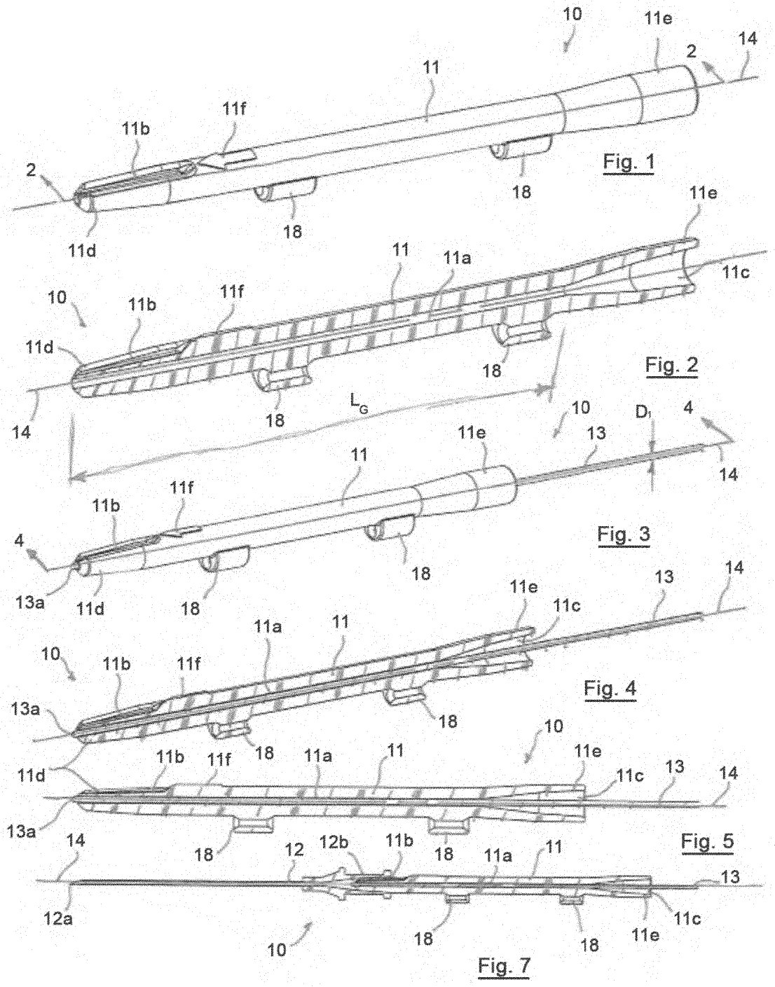

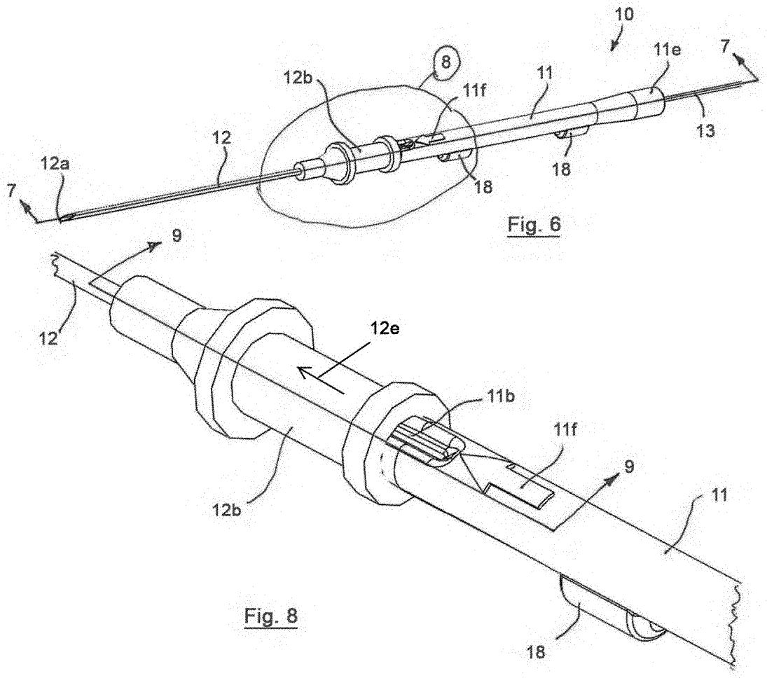

Referring to FIGS. 1-9, a first preferred embodiment of an access device of the present invention, generally designated 10, includes a hub or body 11 with a guidewire channel 11a, flash channel 11b, a distal end 11d, a proximal end 11e and a tapered or funnel-like port 11c at the proximal end 11e extending from the guidewire channel 11a. A guidewire 13 is movably mountable to the body 11, preferably within the guidewire channel 11a, such that a front end 13a of the guidewire 13 is positioned within the guidewire channel 11a in a loaded configuration. The guidewire channel 11a is preferably positioned parallel to and, in the first preferred embodiment, coaxial with a longitudinal axis 14 of the body 11. The funnel-like port or female Luer fitting 11c at the proximal end 11e allows the guidewire channel 11a to be flushed with a standard syringe, if needed to remove blood during multiple access attempts.

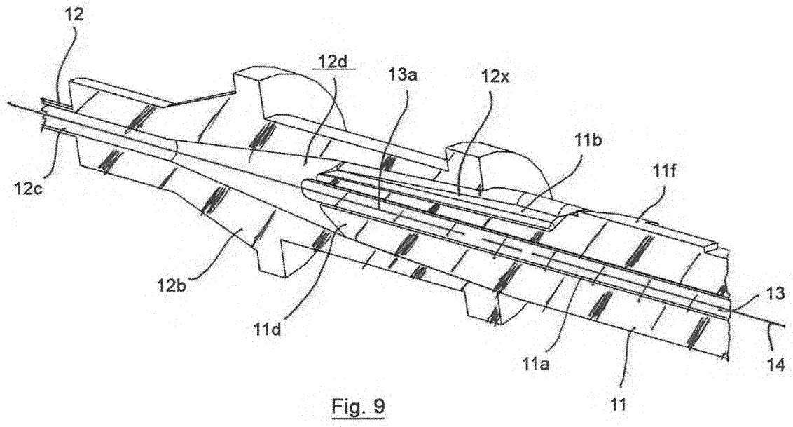

The preferred access device 10 also preferably includes a hypodermic or vascular access needle 12 with a distal tip 12a, a proximal end 12b and a needle lumen 12c. The proximal end 12b is removably mountable to the distal end 11d of the body 11 and the needle lumen 12c is in fluid communication with the guidewire channel 11a and the flash channel 11b in a loaded and assembled configuration. The distal end 11d may be configured in the form of a Luer fitting for connection of the proximal end 12b of the access needle 12 to the body 11 by a press fit, preferably a relatively light press fit. A proximal end 12b of the needle 12 is preferably comprised of a hub housing that is adapted for removable mounting to the distal end 11d of the body 11. The proximal end 12b may be removably attached or secured to the body 11 by a Luer-Lock, threaded connection or other attachment mechanism or method, such as tapering or funnel-like features. The hypodermic or vascular access needle 12 may have different sized and configured needles 12 and may be comprised of a relatively standard hypodermic or vascular access needle 12, such as eighteen gauge, twenty gauge, twenty-one gauge, twenty-two gauge and other sized needles 12. When the needle 12 is mounted to the distal end 11d of the body 11, an inner surface 12d of the proximal end 12b of the needle 12 defines a lumen or flash lumen 12x with the flash channel 11b of the body 11 (FIG. 9). The flash lumen 12x permits flow of blood from within the needle 12 into view of the user at an external, preferably upper surface of the body 11 during use, as will be described in greater detail below. The flash channel is preferably exposed or comprised of a portion of an external surface of the body 11 at or near the distal end 1d and defines the flash lumen 12x with the internal surface of the proximal end 12b of the needle 12. Removing the needle 12 from the body 11, thereby provides access to the flash channel 11b such that the flash channel 11b may be cleaned and/or sterilized by the user after fluid, such as blood flows through the flash lumen 12x.

The body 11 also preferably includes a flash marker 11f on the external surface of the body 11 proximate a proximal end of the flash channel 11b. The flash marker 11f of the first preferred embodiment is comprised of an arrow that points toward the flash channel 11b indicating where a user should expect to see flash or blood flow when a patient's vessel is accessed by the distal tip 12a during use, as will be described in greater detail below. The needle 12 may also include a bevel marker 12e on proximal end 12b that is alignable with the flash marker 11f to align the flash channel 11b with the bevel of the tip 12a. Aligning the bevel of the tip 12a with the flash channel 11b provides consistent insertion of the bevel into the patient and orientation of the flash channel 11b upwardly toward the user so that the flash in readily identified and relatively easily viewed by a user when the vessel is pierced.

In the first preferred embodiment, the body 11 and the needle 12, including the guidewire channel 11a and the needle lumen 12c are positioned on and are coaxial with a longitudinal axis 14 of the access device 10. The needle 12, needle lumen 12c, body 11 and guidewire channel 11a are not limited to being positioned on or coaxial with the longitudinal axis 14, but are so configured in the first preferred embodiment. For example, the needle 12 and guidewire channel 11a may be positioned on or extend substantially parallel to the longitudinal axis 14. The needle lumen 12c preferably extends between the tip 12a and the proximal end 12b and tapers or has a funnel-like shat near the proximal end 12b at the inner surface 12d. The needle lumen 12c is in fluid communication with the flash lumen 12x to facilitate blood flow from the tip 12a, through the flash lumen 12x and out between the body 11 and the proximal end 12b of the needle 12 for observation by the user to indicate that the tip 12a is positioned in the patient's vessel.

The preferred access device 10 may include a guidewire seal or sealing mechanism (not shown) in the guidewire channel 11a, preferably near a distal end of the guidewire channel 11a, that sealingly engages the guidewire 13 in a mounted or loaded configuration. The sealing mechanism preferably limits or prevents fluid flow into the guidewire channel 11a from the needle lumen 12c, but also permits the guidewire 13 to move through the sealing mechanism for introduction into the needle lumen 12c and withdraw of the guidewire 13 from the access device 10. In an initial or loaded configuration, the guidewire 13 is positioned in the guidewire channel 11a such the front end 13a of the guidewire 13 is positioned proximally relative to or within the sealing mechanism and is advanced through the sealing mechanism during use. The guidewire seal preferably limits or prevents blood flow from the needle lumen 12c into the guidewire channel 11a when the guidewire 13 is not loaded in the body 11 and when the guidewire seal engages the guidewire 13 in the loaded configuration or when the guidewire 13 is extended through the guidewire seal. The guidewire seal or sealing mechanism is preferably constructed of an elastic material that permits the guidewire 13 to extend therethrough and collapses on itself when the guidewire 13 is removed from the guidewire channel 11a, although the seal is not so limited. The seal may alternatively be constructed to seal around the guidewire 13 when it is inserted into the guidewire channel 11a and generally not seal the guidewire channel 11a when the guidewire 13 is removed from the guidewire channel 11a or is not in engagement with the guidewire 13.

The guidewire 13 includes the front end portion 13a that is preferably pre-loaded into the guidewire channel 11a in an initial or loaded configuration (FIGS. 4-7 and 9) such that the guidewire 13 is arranged for quick insertion into the patient when the tip 12a is in the vessel, as is described in greater detail below. The front end portion or distal portion 13a of the guidewire has a greater flexibility relative to the remaining length or portion of the guidewire 13, such that the front end portion 13a is able to traverse patient's vessels without puncturing the vessels. In addition, the front end portion 13a may have a curved or J-hooked feature at its distal end that is preferably straightened for insertion into the needle 12 and the patient. The front end portion 13a is approximately one and one-half to two inches (1½-2″). In the preferred embodiment, the guidewire channel length L_{G} is at least two and one-half inches (2½″) or longer such that the front end portion 13a or distal end portion of the guidewire 13 is constrained in the guidewire channel 11a in the loaded configuration, as the guidewire 13 is relatively flexible and may fall out of the guidewire channel 11a without this length or buckle when a user attempts to advance the guidewire 13 into the patient's vessel. The front end portion 13a of the guidewire 13, which has the comparatively high flexibility when compared to the remainder or more proximal portion of the guidewire 13, is, therefore, positioned within the guidewire channel 11a in the loaded configuration with at least some of the more rigid portion of the guidewire 13 also positioned within the guidewire channel 11a such that the guidewire 13 is retained in the body 11 without assistance or active force being applied by the technician. The guidewire 13 is, therefore, generally more stable relative to the body 11 when inserted with the entire front end portion 13a within the guidewire channel 11a, as the more flexible front end portion 13a is contained within the guidewire channel 11a.

A rear end portion or proximal end of the guidewire 13 may be secured relative to the body 11 or may extend freely from the body 11 in the initial or loaded configuration. The rear end portion may be secured to the body 11 by a guidewire holder 18, such as the two guidewire holders 18 of the first preferred embodiment that extend from the body 11 and may receive and secure the rear end portion of the guidewire 13 in the initial configuration. The first preferred access device 10 is not limited to including the guidewire holder 18 shown in the first preferred embodiment and may include an alternative mechanism for securing the rear end of the guidewire 13 to the body 11 or may exclude the guidewire holder 18, without significantly impacting the operation and function of the first preferred access device 10. The guidewire holder 18 (to store rear or proximal end of guidewire 13) preferably prevents the rear end of the guidewire 13 from touching non-sterile objects or from generally uncontrollably moving relative to the body 11 in the loaded configuration.

The first preferred access device 10 can be used to gain access to blood vessels, such as the lumens of arteries and veins. The needle 12 having an appropriate size is connected or secured to the body 11 and the guidewire 13 is pre-loaded onto or into the body 11, preferably in sterile packaging (not shown) associated with the access device 10. The access device 10 may be supplied to a user as a kit, sterile packaged with the body 11, needle 12 and guidewire 13 pre-loaded in the body 11 or may be provided as only the body 11 and the medical professional may select an appropriate needle 12 and guidewire 13 for their preferred procedure. The body 11 preferably has the flash channel 11b that defined the flash lumen 12x with the internal surface 12d of the needle 12 that allow for fluid to pass through the needle 12, into flash lumen 12x and out onto an external surface of the body 11, preferably emerging from the flash channel 11b near the flash marker 11f. The guidewire 13 is preferably pre-loaded such that it can easily be advanced through the needle lumen 12c and into the lumen of a vessel when the top 12a is properly placed in the vessel. The access device 10 is preferably designed such that the guidewire 13 is slidable through the guidewire channel 12a and the needle lumen 12c, thereby allowing the guidewire 13 to be left in the vessel while the needle 12 and the body 11 are completely removed from the area adjacent the patient during the procedure by backing the body 11 and needle 12 proximally away from and off of the guidewire 13.

In the first preferred embodiment, the body 11 includes the single needle 12 secured or fixed thereto, but is not so limited and multiple needles may be secured to the body 11 to secure access to the blood vessel by inserting multiple needles (not shown) into the patient. The needle 12 is preferably removably mountable to the body 11, such as by employing a Luer-Lock connection or other tapered connection that permits mounting of variously sized needles 12 to the distal end 11d of the body 11. The needle 12 is also preferably echogenic or includes an echogenic portion in an area approximately one-half to one centimeter (½-1 cm) from the tip 12a to permit use of the needle 12 with ultrasound for visualization. The echogenic portion may be integrally formed with the needle 12, applied to the needle 12 at the echogenic portion or otherwise positioned at the echogenic portion for visualization purposes. The echogenic portion may alternatively be comprised of a radiopaque marker attached or applied to the needle 12 for visualization using radiant energy techniques and mechanisms, such as x-ray. The echogenic portion may also be positioned at or on the tip 12a. The needle 12 is not limited to including the echogenic portion and may be configured without the echogenic portion, but the echogenic portion is preferred for visualization purposes and may be utilized with any of the preferred access devices 10, 20, 30, 40, 50, 60, 70, 80, described herein. The needle 12 may also include depth markings on an external surface to provide a visual indication of the depth of the tip 12a during insertion into the patient.

The first preferred access device 10 also includes the guidewire channel 11a and the needle lumen 12c positioned generally on or coaxial with the longitudinal axis 14. The guidewire channel 11a and needle lumen 12c are not limited to being positioned on or coaxial with the longitudinal axis 14, as is described in further detail herein.

The guidewire channel 11a of the first preferred embodiment of the access device 10 preferably extends generally from the proximal end 11e to the distal end 11d of the body 11, but is not so limited and may curve or extend at an angle such that the guidewire channel l la does not extend the full length of the body 11. The guidewire channel 11a preferably does extend through the distal end 11d of the body 11 such that the guidewire channel 11a opens into the needle lumen 12c in the initial or assembled configuration. The guidewire 13 may be positioned such that the tip of the front end 13a of the guidewire 13 extends out of the guidewire channel 11a in the loaded or initial configuration for relatively quick extension into the needle lumen 12c when access to the vessel is secured. The guidewire channel 11a preferably defines a channel length LG where an outer surface of the guidewire 13 is positioned close or tightly relative to an inner surface of the guidewire channel. The channel length LG is preferably long enough to secure the relatively flexible front end portion 13a of the guidewire 13 in the loaded configuration. The front end portion 13a may be relatively elastic or flexible relative to the remaining portions of the guidewire 13 to form a J-hook or atraumatic tip of the guidewire 13 in a relaxed configuration. Accordingly, in the loaded configuration, the positioning of the front end portion 13a within the relatively straight or constrained guidewire channel 11a deforms the front end portion 13a from the J-hook or other atraumatic shape. When the front end portion 13a is introduced into the vessel, the J-hook or other shaped atraumatic tip flexes to the relaxed configuration to limit punctures to the vessel by a sharp end of the guidewire 13. The channel length LG is preferably at least two inches (2″), more preferably at least three and one-quarter inches (3¼″), but is not so limited and may be greater than three and one-quarter inches (3¼″) for holding the guidewire 13 in the loaded configuration. The channel length LG is preferably sufficient to stabilize the guidewire 13 within the guidewire channel 11a in the loaded configuration, such that at least a portion of the more rigid proximal portion of the guidewire 13 positioned proximally relative to the front end portion 13a is within the guidewire channel 11a in the loaded configuration. The body 11 is also preferably constructed of a transparent or semi-transparent material, such as a biocompatible, transparent or semi-transparent polymeric material that permits visualization of the guidewire 13 within the guidewire channel 11a by the user. The body 11 is not limited to such constructions and may be constructed of an opaque material, such as a biocompatible metal that is able to take on the size and shape of the preferred body 11 and withstand the normal operating conditions of the body 11. The channel length LG is long enough to fully contain the soft distal end of the guidewire 13 and a portion of the firm main section of the guidewire 13, thereby preventing the guidewire 13 from buckling during advancement through the guidewire channel 11a.

The first preferred access device 10 facilitates an access procedure for gaining access to the patient's vessel, but within a single, integrated device, which is an improvement over known prior art devices and systems, particularly with the adaptability of the distal end 11d of the body 11 that removably accepts different sized needles 12 and accommodates the flash channel 11b on an external surface of the body 11 that may be readily cleaned and/or sterilized. The body 11 is designed such that it is releasably connected to the needle 12 with the pre-loaded guidewire 13 in the guidewire channel 11a.

In operation, the first preferred access device 10 may be packaged in a sterile package or kit with the guidewire 13 loaded in the guidewire channel 11a. The body 11 may alternatively be separately packaged and the user may select separate needles 12 and guidewires 13 for use with the body 11. The access device 10 is removed from the package and the needle 12 may be mounted to the distal end 11d of the body 11, thereby forming the flash lumen 12x. The tip 12a is positioned by the physician or medical professional adjacent the patient's skin near an anatomical region where a predetermined vessel should be located for a particular procedure. The tip 12a is inserted into the skin and the physician or medical professional visually inspects the opening or port of the flash lumen 12x near the flash marking 12f waiting for blood to appear in the flash channel 12x or nearly anywhere on external surfaces of the body 11 and needle 12 as the tip 12a is urged into the patient. The pressure in the blood vessel forces blood flow into the needle lumen 12c, into the flash lumen 12x and out of the flash channel 11b near the tip of the arrow of the flash marker 12f. The guidewire seal preferably seals or partially seals the guidewire channel 11a from the blood flow such that the blood flowing into the needle lumen 12c is urged into the flash lumen 12x. The seal also preferably self-heals or collapses when the guidewire 13 is removed to substantially prevent fluid from flowing out of the guidewire channel 13a. Once access is gained to the blood vessel, front end portion 13a of the guidewire 13 is urged into the needle lumen 12c and into the vessel. The front end portion 13a may move to its relaxed configuration, such as the J-hook configuration or other atraumatic configuration to limit exposure of the vessel to sharp edges of the guidewire 13. When the guidewire 13 is positioned within the vessel, the needle 12 and body 11 may slide proximally along the longitudinal axis 14 away from the patient on the guidewire 13, while the guidewire 13 is retained in the patient and, particularly the lumen of the blood vessel. The guidewire 13 is subsequently used to guide instruments or implants into the vessel for further procedures.

The pre-loaded guidewire 13 with its rear end portion secured to the body 11 at the guidewire holder 18 is inserted through the needle 12 and into the vessel, without requiring the physician or medical technician to reach for and insert the guidewire 13 into the guidewire channel 11a after gaining access to the vessel because of the pre-loading. The guidewire 13 is preloaded in that the front end portion 13a of the guidewire 13 is at least partially positioned in the guidewire channel 11a in the initial or loaded configuration (FIGS. 4-7 and 9). The guidewire 13 is urged into the appropriate vessel and the needle 12 and body 11 are then removed over the guidewire 13 away from the patient with the guidewire 13 remaining in the vessel. The guidewire 13 is then utilized to guide additional devices or testing apparatus to the vessel. The body 11 may be adapted for use with variously sized and shaped needles 12 that are removably mountable to the distal end 11d of the body 11 and facilitate formation of the flash lumen 12x when the needle 12 is mounted to the body 11. In addition, the adaptability of needle sizes facilitates use of variously sized guidewires 13 with the body 11 so that the user or medical professional may select a preferred size for the needle 12 and the guidewire 13 based on preferences or particular procedures.

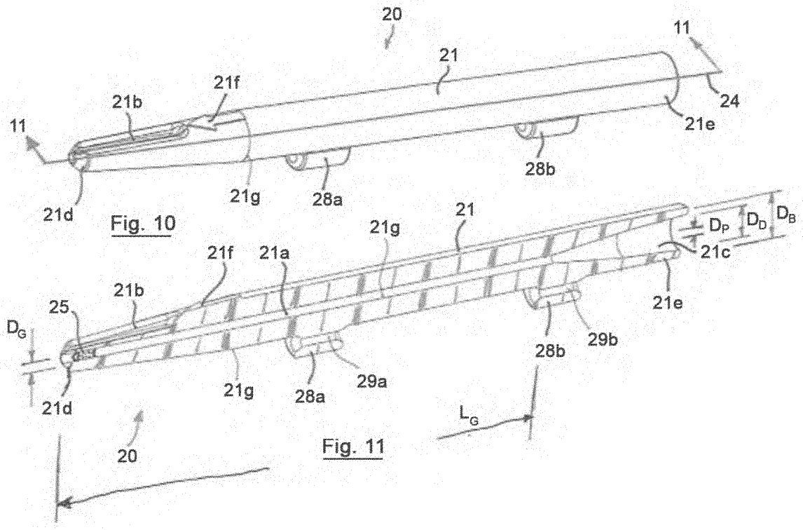

Referring to FIGS. 10 and 11, a second preferred access device 20 has a similar construction to the first preferred access device 10 and like reference numbers are utilized to identify like features of the second preferred access device 20 with a number “2” prefix replacing the “1” prefix to distinguish the features of the access device 10 of the first preferred embodiment from the access device 20 of the second preferred embodiment.

The access device 20 of the second preferred embodiment is configured to access a lumen of a blood vessel. The access device 20 of the second preferred embodiment includes a guidewire seal or sealing mechanism 25 in the guidewire channel 21a near the distal end 21d of the body 21 and near the outlet port of the guidewire channel 21a. The guidewire seal 25 sealingly engages the guidewire (not shown) in a mounted or loaded configuration. The sealing mechanism 25 limits or prevents fluid flow into the guidewire channel 21a from the needle lumen (not shown), but also permits the guidewire to move through the sealing mechanism 25 for introduction into the needle lumen and withdraw of the guidewire from the access device 20. In an initial or loaded configuration, the guidewire 13 is positioned in the guidewire channel 21a such the front end of the guidewire 13 is positioned proximally relative to or within the sealing mechanism 25 and is advanced through the sealing mechanism 25 during use. The guidewire seal 25 preferably limits or prevents blood flow from the needle lumen into the guidewire channel 21a when the guidewire is not loaded in the body 21. When the guidewire seal engages the guidewire 13 in the loaded configuration or when the guidewire 13 is extended through the guidewire seal 25. The guidewire seal 25 of the second preferred embodiment is positioned at or near the outlet port of the guidewire channel 21a, but is not so limited and may be positioned anywhere within the guidewire channel 21a that limits or prevents flow of fluid through the guidewire channel 21a during use. In the initial configuration, the front end portion 13a of the guidewire 13 is positioned proximally or at least partially within the guidewire seal 25, but is not so limited and the access device 20 may be configured without the guidewire seal 25 such that flash flows out of the funnel-like port 21c when the vessel is accessed. For example, the second preferred access device 20 may be configured without the guidewire seal 25 and the guidewire channel 21a having a greater diameter than the guidewire 13, such that blood flow or flash flows past the guidewire 13 through the guidewire channel 21a and out of the funnel-like port 21c to provide a physical and visual indication to the user that the vessel is accessed.

The second preferred access device 20 includes the body 21 having the distal end 21d, the proximal end 21e, the longitudinal axis 24 and the guidewire channel 21a. The guidewire channel 21a extends generally parallel to the longitudinal axis 24 and extends substantially coaxial to the longitudinal axis 24 in the preferred embodiment. The guidewire channel 21a is defined at the distal end 21d of the body 21 and extends toward the proximal end 21e of the body 21. The guidewire channel 21a opens into the funnel-like port 21c at the proximal end 21e. The funnel-like port 21c has a proximal port diameter D_{P} that is greater than a distal port diameter D_{D} such that the opening at the distal end 21d is relatively large for insertion of the guidewire 13. The distal port diameter D_{D} is substantially the same as a guidewire channel diameter D_{G}. The guidewire channel 21a defines a channel length L_{G} that is at least two inches (2″) and more preferably three and one-quarter inched (3¼″) in the second preferred embodiment. The channel length LG is preferably at least two inches (2″) such that the guidewire 13 is retained in guidewire channel 21a in the initial configuration while the needle 12 is inserted into the patient's vessel and while flood flows through the needle 12, past the guidewire 13 through the guidewire channel 21a and out of the funnel-like port 21c at the distal end 21d. The guidewire 13 can then be inserted into the patient's vessel, once access is gained to the vessel, such that a subsequent device can be guided into the vessel on the guidewire 13. The funnel-like portion 21c of the preferred embodiment tapers from the proximal port diameter D_{P} to the disport port diameter D_{D} to provide the expanded diameter for insertion of the front end portion 13a into the guidewire channel 21a. The body 21 of the preferred embodiment also has a substantially consistent body diameter D_{B} between the proximal end 21e and a distal trip transition edge 21g proximate the distal end 21d. The guidewire 13 has a guidewire diameter D₁

The second preferred access device 20 also includes the needle 12 with the tip 12a having the proximal end 12b that is removably mountable to the distal end 21d of the body 21. The guidewire 13 has a guidewire diameter D₁ and the guidewire channel diameter D_{G} is greater than the guidewire diameter D₁. The guidewire channel 21a is configured to facilitate blood flow between the guidewire 13 and an internal surface 21g of the guidewire channel 21a when the guidewire 13 is positioned in the guidewire channel 21a such that the blood flash flows out of the funnel-like port 21c when the vessel is accessed. The body 21 also includes a guidewire holder 28a, 28b extending form a side of the body 21. The guidewire holder 28a, 28b of the second preferred embodiment has a holder channel 29a, 29b configured to secure the rear end portion of the guidewire 13 in the initial configuration such that the rear end portion is not free to move relative to the body 21 during a procedure. The guidewire holder 28a, 28b of the second preferred embodiment includes a proximal guidewire holder 28b with a proximal holder channel 29b and a distal guidewire holder 28a with a distal holder channel 29a. The proximal guidewire holder 28b is positioned closer to the proximal end 21e than the distal guidewire holder 28a.

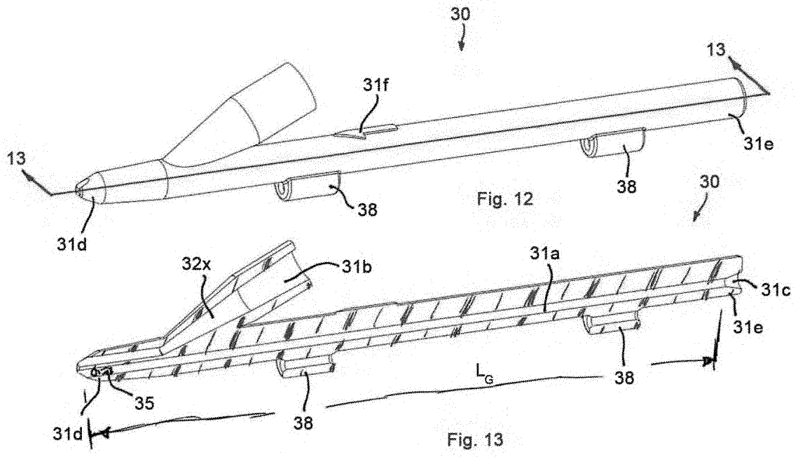

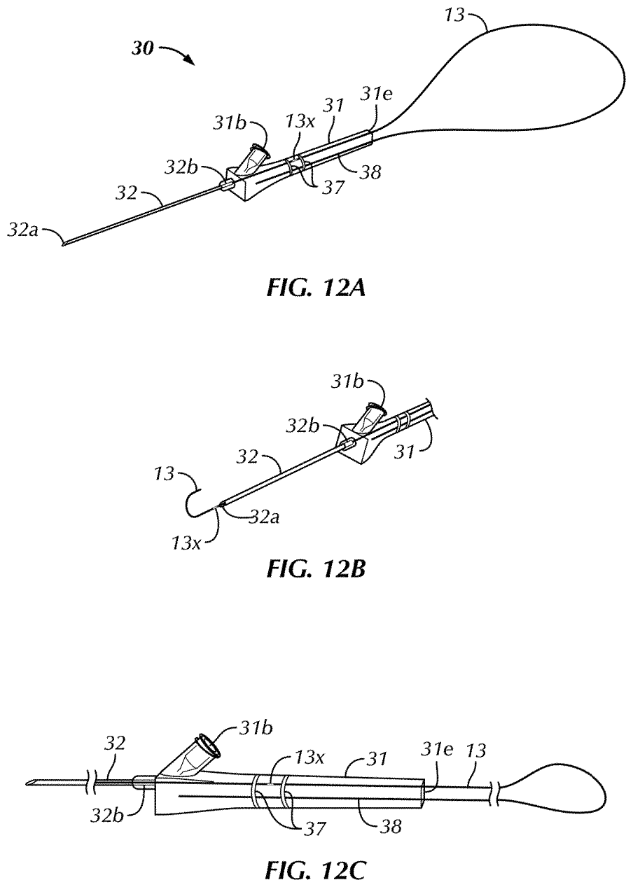

Referring to FIGS. 12-13, a third preferred access device 30 has a similar construction to the first and second preferred access devices 10, 20 and like reference numbers are utilized to identify like features of the third preferred access device 30 with a number “3” prefix replacing the “1” and “2” prefixes to distinguish the features of the access devices 10, 20 of the first and second preferred embodiments from the access device 30 of the third preferred embodiment.

The access device 30 of the third preferred embodiment includes an integrally formed flash port 31b that defines the flash lumen 32x. The distal end 31d of the body 31 is configured for removable mounting to the hypodermic needle 12, preferably hypodermic needles 12 having different sizes and shapes for various procedures. In use, the blood flash flows through the needle lumen (not shown) into the flash lumen 32x and out of the body 31 near the arrowhead of the flash marker 31f.

The body 31 of the third preferred embodiment also includes guidewire alignment markings 37 on an outer surface and the guidewire 13 includes a tip distance marker 13x positioned proximally relative to the front end 13a of the guidewire 13. The tip distance marker 13x is preferably positioned between the guidewire alignment markings 37 in the mounted or loaded configuration (FIGS. 12A and 12C). In the mounted or loaded configuration with the tip distance marker 13x positioned between the guidewire alignment marking 37, the front end 13a is positioned near the distal end of the guidewire channel 31a, but not in the needle lumen (not shown) of the needle 12. Positioning the front end 13a of the guidewire 13 near the distal end of the guidewire channel 31a, but not within the needle lumen permits blood to flow through the needle lumen when the vessel is punctured by the tip 32a while readying the guidewire 13 for relatively quick introduction into the vessel upon identification of blood flash. The guidewire alignment markings 37 are not limited to being comprised of lines on the body 13, the tip distance marker 13x is not limited to being a marking on the guidewire 13 and these components may be comprised of nearly any marker or identification that indicates to the user positioning of the front end 13a of the guidewire 13 near the distal end of the guidewire channel 31a in the loaded configuration. For example, the front end 13a may be positioned in the loaded configuration by aligning a notch (not shown) on the guidewire 13 with an alignment window (not shown) on the body 31.

Referring to FIGS. 12A-12C, the body 31 may include the continuous guidewire holder 38, shown in FIGS. 12A-12C, that receives and holds a rear end portion of the guidewire 13 in the loaded configuration. The guidewire holder 38 of the third preferred embodiment preferably is integrally formed in the body 31 and extends generally parallel to the guidewire channel 31a, but is not so limited and may be otherwise designed and configured to hold the rear end portion of the guidewire 13 in the loaded configuration.

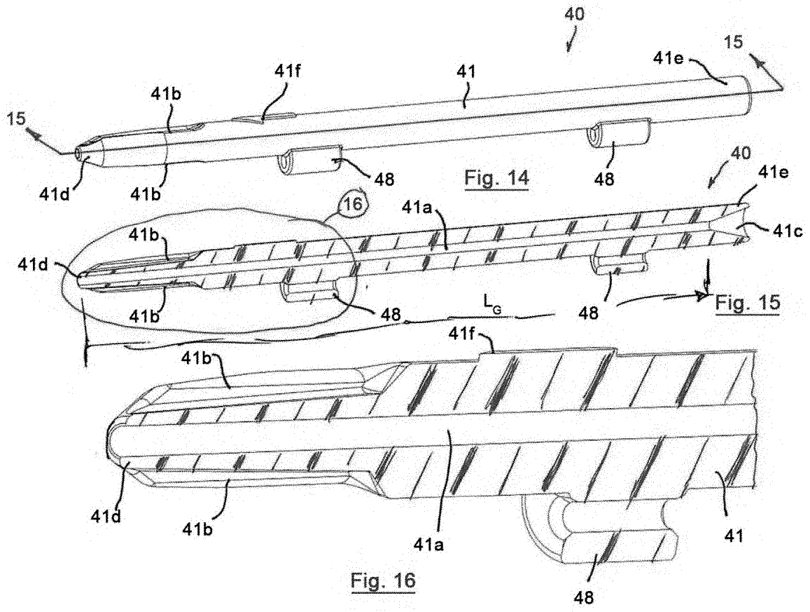

Referring to FIGS. 14-16, a fourth preferred access device 40 has a similar construction to the first, second and third preferred access devices 10, 20, 30 and like reference numbers are utilized to identify like features of the fourth preferred access device 40 with a number “4” prefix replacing the “1,” “2” and “3” prefixes to distinguish the features of the access devices 10, 20, 30 of the first, second and third preferred embodiments from the access device 40 of the fourth preferred embodiment.

The fourth preferred access device 40 includes the body 41 with upper and lower flash channels 41b, 41b at the distal end 41d of the body 41. Both the upper and lower flash channels 41b, 41b define flash lumens with the inner surface 12d of the needle 12 such that blood flashes from upper and lower surfaces of the body 41 at the proximal portions of the upper and lower flash channels 41b, 41b. The upper and lower flash channels 41b, 41b permit visualization of the blood flash by the user regardless of the orientation of the body 41 relative to the user during use.

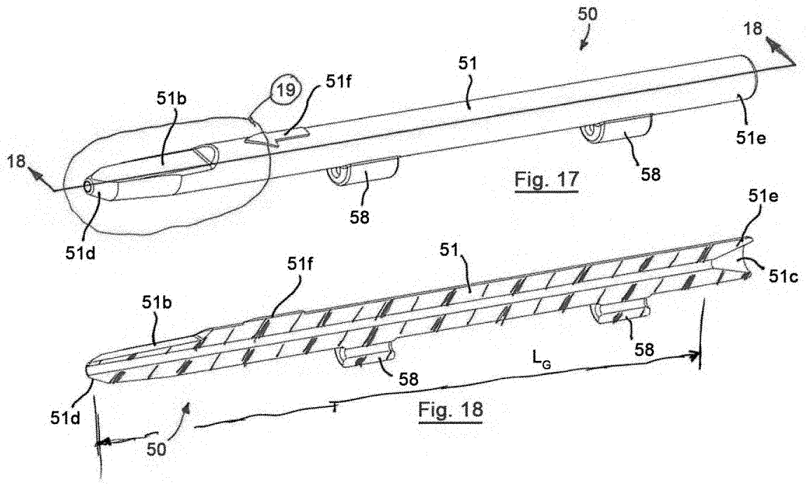

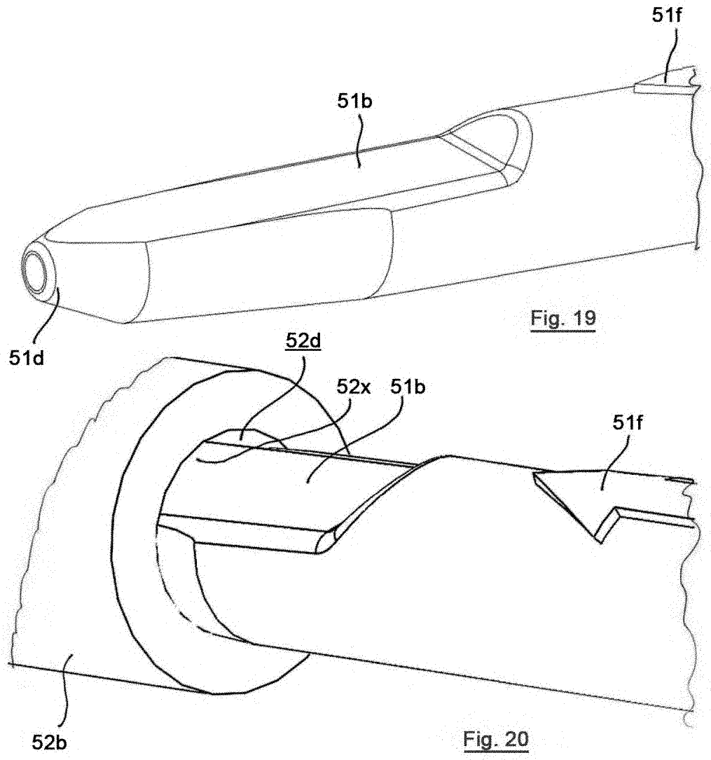

Referring to FIGS. 17-20, a fifth preferred access device 50 has a similar construction to the first, second, third and fourth preferred access devices 10, 20, 30, 40 and like reference numbers are utilized to identify like features of the fifth preferred access device 50 with a number “5” prefix replacing the “1,” “2,” “3” and “4” prefixes to distinguish the features of the access devices 10, 20, 30, 40 of the first, second, third and fourth preferred embodiments from the access device 50 of the fifth preferred embodiment.

In the fifth preferred embodiment, the access device 50 includes a relatively flat flash channel 51b at the distal end 51d that defines the flash lumen 52x with the proximal end 52b of the needle. The flash lumen 52x is defined by the inner surface 52d of the proximal end 52b of the needle and the flash channel 51b of the body 51. The relatively flat flash channel 51b facilitates relatively simple cleaning and sterilization of the flash channel 51b after the needle 12 is removed from the body 51. The distal end 51d is designed and configured for engagement with the multiple hypodermic needles 12 having different shapes and sizes for user preferences or procedures.

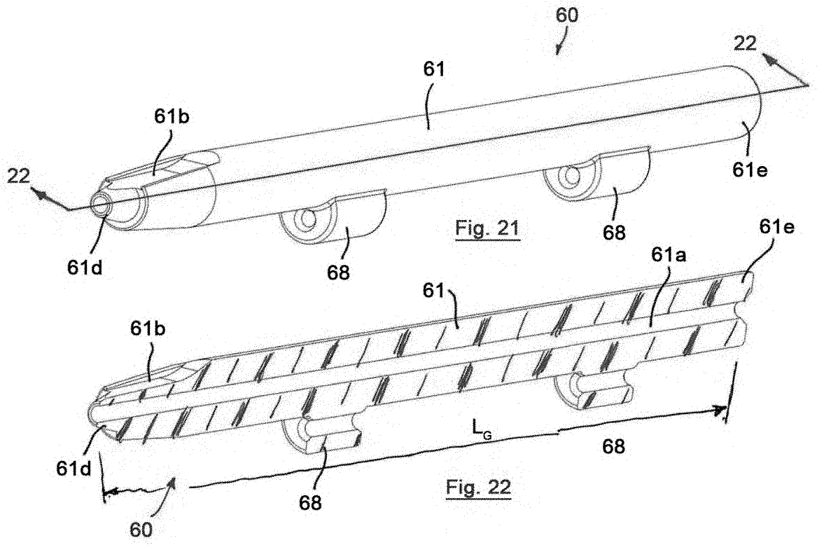

Referring to FIGS. 21 and 22, a sixth preferred access device 60 has a similar construction to the first, second, third, fourth and fifth preferred access devices 10, 20, 30, 40, 50 and like reference numbers are utilized to identify like features of the sixth preferred access device 60 with a number “6” prefix replacing the “1,” “2,” “3,” “4” and “5” prefixes to distinguish the features of the access devices 10, 20, 30, 40, 50 of the first, second, third, fourth and fifth preferred embodiments from the access device 60 of the sixth preferred embodiment.

In the sixth preferred embodiment, the access device 60 includes a slightly wider flash channel 51b with end walls and does not include a flash marker at the distal end 51d.

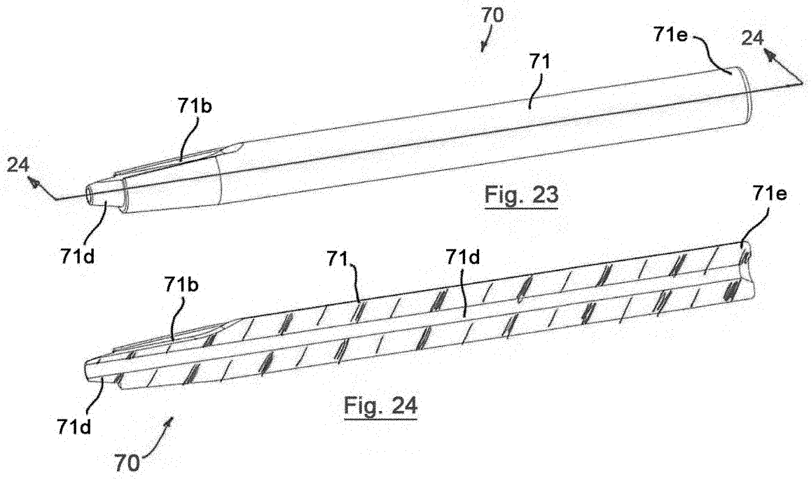

Referring to FIGS. 23 and 24, a seventh preferred access device 70 has a similar construction to the first, second, third, fourth, fifth and sixth preferred access devices 10, 20, 30, 40, 50, 60 and like reference numbers are utilized to identify like features of the seventh preferred access device 70 with a number “7” prefix replacing the “1,” “2,” “3,” “4,” “5” and “6” prefixes to distinguish the features of the access devices 10, 20, 30, 40, 50, 60 of the first, second, third, fourth, fifth and sixth preferred embodiments from the access device 70 of the seventh preferred embodiment.

The seventh preferred body 71 has a generally cylindrical-shape from the distal end 71d to the proximal end 71e without guidewire holders on the outer surface of the body 71. The body 71 also does not include a flash marker on the external surface, but is not so limited.

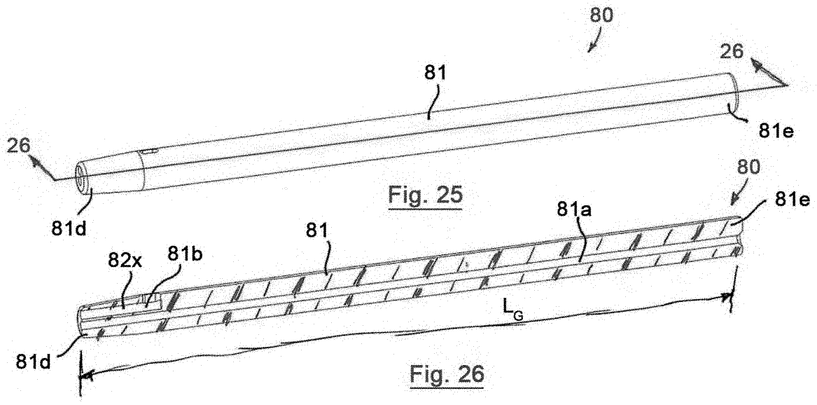

Referring to FIGS. 25 and 26, an eighth preferred access device 80 has a similar construction to the first, second, third, fourth, fifth, sixth and seventh preferred access devices 10, 20, 30, 40, 50, 60, 70 and like reference numbers are utilized to identify like features of the eighth preferred access device 80 with a number “8” prefix replacing the “1,” “2,” “3,” “4,” “5,” “6” and “7” prefixes to distinguish the features of the access devices 10, 20, 30, 40, 50, 60, 70 of the first, second, third, fourth, fifth, sixth and seventh preferred embodiments from the access device 80 of the eighth preferred embodiment.

The eighth preferred body 81 also has a generally cylindrical-shape from the distal end 81d to the proximal end 81e and also includes a flash lumen 82x that is integrally defined at the distal end 81d of the body 81. The distal end 81d is configured to removably engage the multiple needles 12 for flexibility of the access device 80.

Referring to FIGS. 1-26, the preferred access devices 10, 20, 30, 40, 50, 60, 70, 80 are an accessory that may connect to a standard vascular access or hypodermic needle 12. The preferred access devices 10, 20, 30, 40, 50, 60, 70, 80 also receive a standard guidewire 13 (i.e. 0.018″, 0.025″, 0.035″, 0.038″) and can be offered to be compatible with a range of needle sizes (i.e. 18 ga, 20 ga, 21 ga, 22 ga).

In use, when the tip 12a of the needle 12 is inserted through the skin into a blood vessel of the patient, the blood passes through the needle and exits from the flash port at the end of the flash lumen 12x, 32x, 52x, 82x indicating the needle 12 is in the blood vessel. The preloaded guidewire 13 is then immediately advanced through the guidewire channel 11a, 21a, 31a, 41a, 51a, 61a, 71a, 81a and the lumen 12c of the needle 12 and into the blood vessel.

In a preferred example, a standard thirty-five thousandths of an inch (0.035″) or fifty centimeter (50 cm) J-wire is commonly used during the Seldinger technique. The J-tip of the guidewire 13 is preloaded in the guidewire channel 11a, 21a, 31a, 41a, 51a, 61a, 71a, 81a so the user doesn't have to fumble with getting the J-wire into the body 11, 21, 31, 41, 51, 61, 71, 81 in the delicate moment after the flash of blood has been observed. Once the wire 13 is advance through the needle 12 and exits into the blood vessel, the J-tip resumes its shape, protecting the blood vessel from damage (i.e. perforation). When loaded into the guidewire channel 11a, 21a, 31a, 41a, 51a, 61a, 71a, 81a the J-tip of the guidewire 13 can't return to its J-shape in the relaxed configuration until the J-tip exits the distal tip 12a of the needle 12. The J-tip of the guidewire 13 also doesn't have enough space between the distal end 11d, 21d, 31d, 41d, 51d, 61d, 71d, 81d of body 11, 21, 31, 41, 51, 61, 71, 81 and the inside of the hub of the needle 12 to resume the J-shape

The male Luer fitting or cone angle of the distal end 11d, 21d, 31d, 41d, 51d, 61d, 71d, 81d allows the body 11, 21, 31, 41, 51, 61, 71, 81 to stay connected to a standard needle Luer fitting of the needle 12 with a light press fit.

In the preferred embodiment, the body 11, 21, 31, 41, 51, 61, 71, 81 is constructed of a clear polymeric or plastic material, more preferably a polycarbonate material, so the user can visualize the guidewire 13 at all times during use. The preferred access devices 10, 20, 30, 40, 50, 60, 70, 80 are lightweight so that the balance of the needle 12 is not disturbed during use. The preferred bodies 11, 21, 31, 41, 51, 61, 71, 81 can be easily disconnected from the needle 12 and its preferred Luer fitting if desired. The preferred guidewire channel 11a, 21a, 31a, 41a, 51a, 61a, 71a, 81a can be flushed using a standard syringe at the proximal end, if needed. The trailing end of the guidewire 13 can be inserted into the guidewire holder 18 to prevent the trailing end from touching a non-sterile object during use and from generally flopping around during use.

It will be appreciated by those skilled in the art that changes could be made to the embodiments described above without departing from the broad inventive concept thereof. For example, the features of each of the preferred embodiments of the access devices 10, 20, 30, 40, 50, 60, 70, 80 may be mixed and matched based on user preferences and for particular procedures. It is understood, therefore, that this invention is not limited to the particular embodiments disclosed, but is intended to cover modifications within the spirit and scope of the present invention as defined by the present disclosure.

We claim:

1. An access device for accessing a lumen of a blood vessel, the access device comprising:
a body having a distal end, a proximal end, a longitudinal axis and a guidewire channel, the guidewire channel extending generally parallel to the longitudinal axis, the guidewire channel defined at the distal end of the body and extending toward the proximal end of the body, the guidewire channel opening into a port positioned at the proximal end of the body, the guidewire channel defines a guidewire channel length that is at least two and one-half inches;
a needle having a tip, a proximal end and a needle lumen extending to the tip, the proximal end of the needle having a funnel-like port with an internal surface, the proximal end secured to the distal end of the body in an assembled configuration; and
a guidewire having a front end portion, the front end portion positioned in the guidewire channel in an initial configuration, the guidewire having a guidewire diameter, the guidewire channel diameter being greater than the guidewire diameter, the guidewire channel configured to facilitate blood flow between the guidewire and an internal surface of the guidewire channel when the guidewire is positioned in the guidewire channel.

2. The access device of claim 1, further comprising:
a flash channel formed at the distal end of the body, the flash channel extending toward the proximal end to a flash marker.

3. The access device of claim 2, wherein the flash marker is comprised of an arrow that points toward the flash channel.

4. The access device of claim 1, further comprising:
a guidewire holder extending from a side of the body, the guidewire holder having a holder channel configured to secure a rear end portion of the guidewire in an initial configuration.

5. The access device of claim 4, wherein the guidewire holder includes a proximal guidewire holder and a distal guidewire holder, the proximal guidewire holder positioned closer to the proximal end than the distal guidewire holder.

6. The access device of claim 1, wherein the guidewire channel length is approximately three inches.

7. The access device of claim 1, wherein the channel length is at least three and one-quarter inches.

8. The access device of claim 1, further comprising:
a guidewire seal positioned near the distal end in the guidewire channel.

9. The access device of claim 8, wherein the front end portion is positioned proximally or at least partially within the guidewire seal in an initial configuration.

10. The access device of claim 8, wherein the guidewire seal is positioned near an outlet port of the guidewire channel.

11. The access device of claim 1, wherein the port of the guidewire channel is comprised of a funnel-like port having a proximal port diameter that is greater than a distal port diameter, the distal port diameter being substantially the same as a guidewire channel diameter.

12. The access device of claim 11, wherein the funnel-like port tapers from the proximal port diameter to the distal port diameter.

13. The access device of claim 1, wherein the body has a substantially consistent body diameter between the proximal end and a distal tip transition edge.

14. The access device of claim 1, wherein the distal end of the body is comprised of a male slip Luer.

15. The access device of claim 1, wherein the proximal end of the needle is removably secured to the distal end of the body in the assembled configuration.

16. The access device of claim 1, wherein the proximal end of the needle is fixedly secured to the distal end of the body in the assembled configuration.

17. The access device of claim 1, wherein the port at the proximal end of the body is comprised of a female Luer connection.
